(19) United States
(12) Patent Application Publication
(10) Pub. No.: US 2019/0184909 A1
(43) Pub. Date: Jun. 20, 2019

(54) Photographing Device Mounted on Vehicle and Photographing Method in Vehicle

(71) Applicant: Yazaki Corporation, Tokyo (JP)

(72) Inventors: Takashi Shiota, Susono-shi (JP); Kenichi Nagahashi, Susono-shi (JP)

(73) Assignee: Yazaki Corporation, Tokyo (JP)

(21) Appl. No.: 16/218,974

(22) Filed: Dec. 13, 2018

(30) Foreign Application Priority Data
Dec. 14, 2017 (JP) 2017-239722

(51) Int. Cl. B60R 11/04; A61B 5/18; B60W 40/09; B60K 28/06; G08B 21/06; A61B 5/00

(52) U.S. Cl. CPC B60R 11/04; A61B 5/18; B60W 40/09; B60W 2040/0872; G08B 21/06; A61B 5/6893; B60K 28/06

(57) ABSTRACT

A photographing device mounted on a vehicle includes a photographing part which is mounted on the vehicle to photograph a face of a driver, a wheel position adjusting part which adjusts a position of a steering wheel mounted on the vehicle, a photographing position adjusting part which adjusts at least one of a photographing position of the photographing part and a photographing angle of the photographing part, and a position adjusting control part which controls at least one of the wheel position adjusting part and the photographing position adjusting part based on a photographed result of the photographing part.

### CROSS REFERENCE TO RELATED APPLICATIONS

This application claims priority from Japanese Patent Application No. 2017-239722 filed on Dec. 14, 2017, the entire contents of which are incorporated herein by reference.

### BACKGROUND OF THE INVENTION

### Field of the Invention

The present invention relates to a photographing device mounted on a vehicle and a photographing method in a vehicle.

### Description of Related Art

A vehicle safety technique is developed that the face of a driver who is driving a vehicle is photographed to monitor sleepiness, fatigue and a looking-away driving of the driver and ensure the safety of the vehicle.

For instance, Patent Literature 1 discloses, as a device which realizes the vehicle safety technique for detecting the sleepiness or the fatigue of the driver, a device having a photographing unit (a camera) which photographs an image to be photographed including the face of the driver to estimate a degree of awakening of the driver based on an image photographed by the camera.

Further, Patent Literature 2 discloses a device including a photographing unit (a camera) which photographs the eye of a driver, a line of vision detection unit which detects the line of vision of the driver from a photographed image of the camera and a movement of line of vision measuring unit which measures the movement of the line of vision of the driver in order to prevent the looking-away driving of the driver, in which a frequency that the line of vision of the driver directed to a preset carefully watching direction is lower than a frequency as a reference, an alarm is raised that a visual recognition of the driver is insufficient.

[Patent Literature 1] JP-A-2008-167806

[Patent Literature 2] JP-A-2006-224700

In a photographing device mounted on a vehicle, a face of a driver needs to be assuredly caught and photographed. According to a related art, when the face of the driver cannot be caught, a driving state of a driver such as sleepiness, fatigue and the looking-away driving of the driver cannot be monitored by a device. Thus, the device hardly contributes to the safety of the vehicle. Further, when a camera is mounted on a meter panel, a steering wheel is provided between the camera and the driver. Accordingly, the camera photographs the face of the driver through an opening part of the steering wheel. However, according to circumstances, the steering wheel may possibly form a dead angle.

Further, a study or a development of a mechanism or a function which is mounted on a vehicle to control or support an automatic driving is advanced. For instance, an electronic control unit (ECU) of the vehicle is constructed so as to meet an automatic driving of a level 2 (LV2) or a level 3 (LV3) of an automation level prescribed by the Japanese government or US Ministry of Transport Road Traffic Safety Bureau (NHTSA). In the level 2, a system automatically carries out a plurality of operations of acceleration, a steering and a brake of the vehicle. In the level 3, since the system carries out all the controls of the acceleration, the steering and the brake of the vehicle, the driver may ordinarily merely monitor a state of the driving.

However, even in the level 3, when the system makes a request at the time of emergency or limitation of the system, the driver must hand over (H/O) the automatic driving to a manual operation in accordance with this request. Accordingly, the photographing device mounted on the vehicle always needs to catch the face of the driver even in the level 3 during a shift to the level 2 from the level 3, namely, in preparation for the hand over. Then, in the level 3, it is well supposed that the driver cannot completely rest, however, the driver relaxes more than an ordinary time, so that a seated position may be possibly loose.

Here, when the face of the driver is located at a standard position even in the level 3, the camera can catch the face of the driver even at the time of the hand over. However, as described above, when the driver relaxes in the level 3 and the position of the face is excessively changed, the face may possibly deviate from a photographing range of the camera depending on the position of the camera or the opening part of the steering wheel, so that the face may not be possibly photographed by the usual photographing device mounted on the vehicle. In this case, the photographing device mounted on the vehicle cannot exhibit its function and may not possibly carry out a hand over at the time of emergency.

Further, according to a study, when the photographing device mounted on the vehicle judges that the face of the driver cannot be photographed, an alarm is given by a lamp or an audio as a fail-safe function. However, the automatic driving which is originally to be comfortable for the driver may be possibly complicated.

### SUMMARY

One or more embodiments provide a photographing device mounted on a vehicle and a photographing method in a vehicle by which the face of a driver can be assuredly caught and photographed.

In an aspect (1), one or more embodiments provide a photographing device mounted on a vehicle, including a photographing part which is mounted on the vehicle to photograph a face of a driver, a wheel position adjusting part which adjusts a position of a steering wheel mounted on the vehicle, a photographing position adjusting part which adjusts at least one of a photographing position of the photographing part and a photographing angle of the photographing part, and a position adjusting control part which controls at least one of the wheel position adjusting part and the photographing position adjusting part based on a photographed result of the photographing part.

In an aspect (2), the photographing device mounted on the vehicle, further includes an image generating part which generates a photographed image based on the photographed result of the photographing part and an image judging part which judges whether or not the face of the driver is included in the photographed image based on the photographed image of the image generating part. The position adjusting control part controls at least one of the wheel position adjusting part and the photographing position adjusting part so as to include the face of the driver in the photographed image based on the judged result of the image judging part.

In an aspect (3), the position adjusting control part compares the photographed image at present with the photographed image in a past to detect a displacement of the face of the driver and control at least one of the wheel position adjusting part and the photographing position adjusting part based on the displacement.

In an aspect (4), a photographing method in a vehicle, includes photographing a face of a driver in a photographing part mounted on the vehicle, generating a photographed image based on a photographed result in the photographing, judging whether or not the face of the driver is included in the photographed image, and adjusting at least one of a position of a steering wheel mounted on the vehicle, a photographing position of the photographing part, and a photographing angle of the photographing part so as to include the face of the driver in the photographed image based on a judged result in the judging.

In an aspect (5), the adjusting includes comparing the photographed images of two times in a past with each other to detect a displacement of the face of the driver and adjusting at least one of the position of the steering wheel, the photographing position of the photographing part, and the photographing angle of the photographing part based on the displacement.

According to the aspect (1), even when a position of the face of the driver is excessively changed, the face of the driver can be caught by a photographing part. Namely, according to the photographing de vice mounted on the vehicle of the present invention, a position adjusting control part is provided which controls at least one of a wheel position adjusting part and a photographing position adjusting part based on the photographed result of the photographing part. Thus, even when the position of the face of the driver is excessively changed, the face of the driver can be caught by the photographing part. Further, a dead angle range of the photographing part is reduced, so that it is rarely necessary to inform of an alarm that a photographing operation cannot be made. Accordingly, since the driver does not need to constantly care about a seated position, a comfortableness of the driver in the vehicle is not harmed.

According to the aspect (2), it is judged whether or not the face of the driver is included in a photographed image to adjust at least one of a position of a steering wheel, a photographing position of the photographing part and a photographing angle of the photographing part so as to include a face of the driver in the photographed image. Thus, even when the position of the face of the driver is excessively changed, the face of the driver can be efficiently and self-consciously caught.

According to the aspect (3), a present photographed image is compared with a past photographed image to detect a displacement of the face of the driver and adjust at least one of the positions of the steering wheel, the photographing position of the photographing part and the photographing angle of the photographing part. Thus, even when the position of the face of the driver is excessively changed, the face of the driver can be rapidly caught by the photographing part.

According to the aspect (4), even when the position of the face of the driver is excessively changed, the face of the driver can be caught by the photographing part. Further, a dead angle range of the photographing part is reduced, so that it is rarely necessary to inform of an alarm that a photographing operation cannot be made. Accordingly, since the driver does not need to constantly care about a seated position, a comfortableness of the driver in the vehicle is not harmed.

Further, it is judged whether or not the face of the driver is included in a photographed image to adjust at least one of a position of a steering wheel, a photographing position of the photographing part and a photographing angle of the photographing part so as to include a face of the driver in the photographed image. Accordingly, even when the position of the face of the driver is excessively changed, the face of the driver can be efficiently and self-consciously caught.

According to the aspect (5), photographed images of two times in the past are compared with each other to detect a displacement of the face of the driver and adjust at least one of the position of the steering wheel, the photographing position of the photographing part and the photographing angle of the photographing part. Thus, even when the position of the face of the driver is excessively changed, the face of the driver can be rapidly caught by the photographing part.

According to one or more embodiments, even when the position of the face of the driver is excessively changed, the face of the driver can be caught by the photographing part. Accordingly, the photographing device mounted on the vehicle and the photographing method in the vehicle of the present invention can be applied to a driving support system which monitors a driving state such as the sleepiness, the fatigue and the looking-away driving of the driver who is driving the vehicle to prevent an erroneous start of the vehicle due to, for instance, a mistake in a driving operation. Especially, in the vehicle on which an automatic driving function is mounted, the photographing device mounted on the vehicle and the photographing method in the vehicle of the present invention are most suitably applied before and after the hand over in which a state that a degree of relation of the driver to the automatic driving is low shifts to a state that a degree of relation of the driver thereto is high.

The present invention is briefly described above. Further, when a mode for carrying out the invention which will be described below is read by referring to the attached drawings, a detail of the present invention will be more clarified.

### BRIEF DESCRIPTION OF THE DRAWINGS

FIG. 1 is a side view showing a driver in a vehicle on which a photographing device mounted on a vehicle according to an exemplary embodiment of the present invention is mounted and main components in front of the driver, which are seen from a left side.

FIG. 2 is a front view showing the vicinity of a meter unit arranged in an instrument panel in front of a driver's seat shown in FIG. 1, which is seen from a direction of the visual point of the driver.

FIG. 3 is a schematic view showing a structure example of a wheel position adjusting mechanism of a steering wheel.

FIG. 4 is a schematic view showing a structure example of a photographing position adjusting mechanism of a camera.

FIG. 5 is a block diagram showing a structure example of a hardware structure of the photographing device mounted on the vehicle according to the exemplary embodiment of the present invention.

FIG. 6 is a functional block diagram showing functions realized by the photographing device mounted on the vehicle shown in FIG. 5.

FIG. 7 is a flowchart showing processes carried out by the photographing device mounted on the vehicle shown in FIG. 5.

### DETAILED DESCRIPTION

Specific exemplary embodiments of the present invention will be described below by referring to the drawings.

### Photographing Device Mounted on Vehicle and Summary of Automatic Driving

Before a specific explanation is made, initially, the summary of a photographing device mounted on a vehicle of the present invention and an automatic driving technique will be described below.

The photographing device mounted on the vehicle photographs a driver by a camera (a photographing part) to detect a driving state such as a behavior or an operation of the driver who drives the vehicle. Then, the photographing device mounted on the vehicle is a device as a base to monitor the driver in a real time based on a photographed image, raise an alarm when an abnormality occurs or automatically record the obtained photographed image.

For instance, the photographing device mounted on the vehicle is used to detect a sleepiness or fatigue of the driver, detect a looking-away driving based on the line of vision of the driver and prevent an unintentional erroneous start of the vehicle by an erroneous operation during a driving.

Further, functions such as the above-described real time monitor, the abnormality alarm and the automatic record can be realized by combining various kinds of known techniques or existing devices with the device of the present invention. For instance, when the photographing device mounted on the vehicle is connected to a drive recorder or a digital tachograph, various kinds of information obtained during the driving of the vehicle can be automatically recorded. Further, when a safety manager of a transportation company or a taxi company analyzes a recorded result of the drive recorder after the driving is finished, the safety manager can guide a safety driving respectively to the crew who gets on the vehicle as the driver.

Further, in recent years, in order to control an automatic driving of the vehicle, a study or development progresses in preparation for an electronic control unit (ECU) of the vehicle in which functions meeting the automatic driving of a level 2 (LV2) or a level 3 (LV3) of an automation level prescribed by, for instance, the Japanese government or US Ministry of Transport Road Traffic Safety Bureau (NHTSA) are mounted on the vehicle.

In the level 2, a system automatically carries out a plurality of operations of acceleration, a steering and a brake of the vehicle. However, in the level 2, the driver constantly needs to monitor a driving state and carry out a driving operation as required.

On the other hand, in the level 3, since the system carries out all the controls of the acceleration, the steering and the brake of the vehicle, the driver may ordinarily merely monitor the state of the driving. Further, ordinarily, the driver does not necessarily need to monitor the driving state.

However, even in the level 3, when the system makes a request at the time of emergency or limitation of the system, the driver needs to respond to this request. Namely, a responsibility for driving needs to be handed over (H/O) to a manual operation of the driver from the system and to shift the level 3 to the level 2 an automation rate of which is lower than that of the level 3. Further, even in the same level, during the automatic driving of the vehicle, an occasion may possibly occur that a traffic line of the vehicle begins to be changed by starting a winker operation, in which the driver is obliged to accompany a hand to a steering wheel and make a monitoring operation. That is, such an occasion may exist that a traveling state in which an assistance of the driver such as an admission of the driver to a decision of the system is not necessary shifts to a traveling state in which any of assistances is necessary. Namely, even in the vehicle on which the automatic driving technique is mounted, an occasion may possibly arise during the driving that a first state in which a driving control is automatically carried out shifts to a second state in which a degree of relation of the driver to the driving control is higher than that of the first state.

As described above, with such kind of photographing device mounted on the vehicle, even in the vehicle on which the automatic driving technique is mounted, it is decisively important to constantly catch the face of the driver and photograph the face. However, the usual photographing device mounted on the vehicle may not possibly meet an occasion that the driver relaxes, for instance, during a completely automatic driving in the level 3 and the position of the face is excessively changed. Namely, in the usual photographing device mounted on the vehicle, the face of the driver may possibly deviate from a photographing range of the camera depending on the position of the camera or the opening part of the steering wheel, so that the face of the driver may not be possibly photographed.

The photographing device mounted on the vehicle according to the present invention has a special structure as in the below-described exemplary embodiment to assuredly photograph the face of the driver.

### Main Components of Photographing Device Mounted on Vehicle and Arrangement Thereof

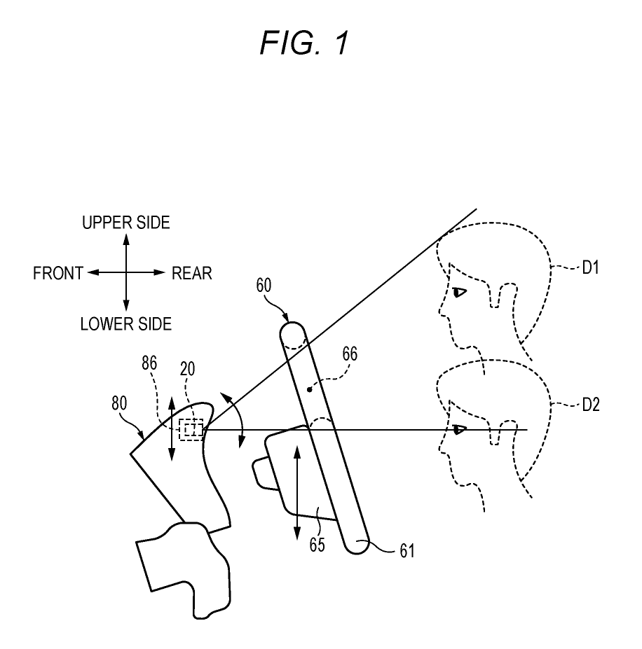

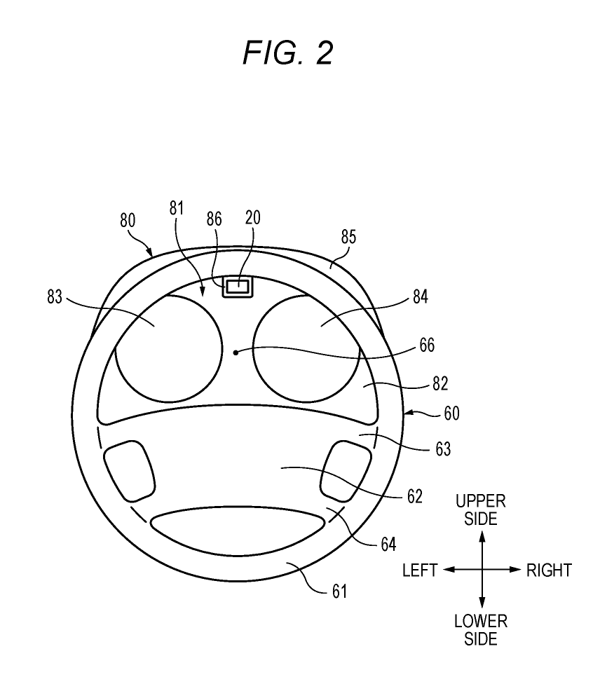

By referring to FIG. 1 and FIG. 2, main components of a photographing device 10 mounted on a vehicle of a present exemplary embodiment and an arrangement of them will be described below. FIG. 1 is a side view showing drivers D1 and D2 in the vehicle on which the photographing device 10 mounted on the vehicle according to the exemplary embodiment of the present invention is mounted and the main components in front of the drivers, which are seen from a left side. FIG. 2 is a front view showing the vicinity of a meter unit 81 arranged in an instrument panel 80 in front of a driver's seat shown in FIG. 1, which is seen from directions of the visual points of the drivers D1 and D2.

In images of two persons shown by dotted lines in FIG. 1, the driver D1 shows an ordinary position of the face during a driving. The driver D2 shows a position of the face when the driver relaxes during the automatic driving (for instance, the level 3) so that the face is located extremely downward. At the time of an actual riding, one driver is seated in the vehicle.

As shown in FIG. 1 and FIG. 2, in front of the drivers D1 and D2 seated in the driver' s seat of the vehicle, a steering wheel 60 and the instrument panel 80 are arranged.

As shown in FIG. 2, the steering wheel 60 has a substantially annular ring part 61, a boss part 62 arranged in a central part of the ring part 61 and connected to a tubular column part 65 so as to freely rotate and two pairs of spoke parts 63 and 64 which diametrically connect the ring part 61 to the boss part 62. Further, the two pairs of spoke parts 63 and 64 include one pair of upper spoke parts 63 extended and arranged in a transverse direction in an upper side of the vehicle and one pair of lower spoke parts 64 extended and arranged in a transverse direction in a lower side of the vehicle. Further, through an opening part 66 defined by the ring part 61, the boss part 62 and the one pair of spoke parts 63 and 63, a below-described camera (a photographing part) 20 of the photographing device 10 mounted on the vehicle catches the faces of the drivers D1 and D2 and photographs the faces (see FIG. 1).

The instrument panel 80 has the meter unit 81. The meter unit 81 includes various kinds of meters or display parts such as a speed meter 83, an engine revolution counter 84, a direction indicating and display part (not shown in the drawing) on its meter panel 82.

As shown in FIG. 1 and FIG. 2, in an upper part and a side part of the meter panel 82, a substantially arc shaped meter hood 85 is provided to extend rearward of the vehicle. The meter hood 85 covers the upper part and the side part of the meter panel 82 to prevent external lights such as solar lights from being directly incident on the meter panel 82. By preventing the incidence, the visibility of the drivers D1 and D2 to the meters or the display parts is ensured.

Further, the instrument panel 80 has a camera attaching part 86 inside of a substantially central position of the meter hood 85. The camera attaching part 86 is provided to protrude downward of the vehicle from the meter hood 85.

The camera 20 is arranged in the vehicle. The camera 20 is embedded in the camera attaching part 86 of the meter hood 85 to photograph the drivers D1 and D2.

Here, as shown in FIG. 1, since an outer upper end part of the boss part 62 of the steering wheel 60 forms an obstacle between the camera 20 and the driver D2 having a position of the face lowered, so that the camera 20 has a dead angle area. Namely, in the position of the opening part 66 of the steering wheel 60, a photographing position of the camera 20 and a photographing angle of the camera 20 shown in FIG. 1, the face of the driver D1 can be photographed by the camera 20, however, the face of the driver D2 cannot be photographed. Accordingly, as described below, the photographing device 10 mounted on the vehicle of the present exemplary embodiment operates a wheel position adjusting mechanism 70 which mechanically adjusts the position of the steering wheel 60 and a photographing position adjusting mechanism (a photographing position adjusting part) which adjusts the photographing position and the photographing angle of the camera 20. Thus, even when a seated position of the driver D2 becomes loose and the position of the face is excessively changed during the automatic driving, the position of the steering wheel 60, the photographing position of the camera 20 and the photographing angle of the camera 20 are adjusted based on the change to catch the face of the driver D2.

### Structure of Wheel Position Adjusting Mechanism

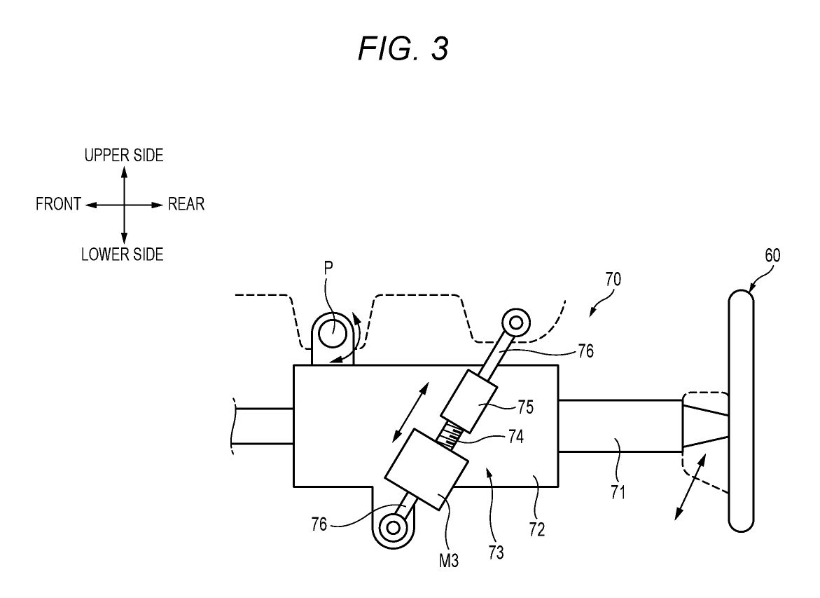

FIG. 3 is a schematic view showing a structure example of a wheel position adjusting mechanism 70 of the steering wheel 60 shown in FIG. 1. As shown in FIG. 3, the wheel position adjusting mechanism 70 of the steering wheel 60 serves to adjust a vertical (tilt) direction of the steering wheel 60 in the vehicle, and includes a substantially cylindrical housing 72 which supports a steering shaft 71 so as to be freely swung on a swing pivot P relative to a vehicle body B of the vehicle and a straight moving mechanism 73 connected to the housing 72 and the vehicle body B so as to be freely swung.

The wheel position adjusting mechanism 70 is ordinarily used to adjust the position of the steering wheel 60 to obtain a maneuverability of steering based on the physical build of the drivers D1 and D2. The wheel position adjusting mechanism is provided as a standard for the first purpose of improvement of the maneuverability of steering depending on vehicles. Accordingly, in the present exemplary embodiment, when the wheel position adjusting mechanism 70 is mounted on the vehicle as a standard, an existing mechanism thereof is commonly used.

In the steering shaft 71, the steering wheel 60 is concentrically attached to a rear end part thereof. To a front end part thereof, a steering mechanism not shown in the drawing is connected. The steering wheel 60 is operated by the drivers D1 and D2, so that steering wheels are steered and rotated through the steering mechanism.

The straight moving mechanism 73 includes a wheel position adjusting motor M3 which is driven to rotate in accordance with a command of a below-described control circuit 40 of the photographing device 10 mounted on the vehicle, a screw shaft 74 driven to rotate by the wheel position adjusting motor M3, a nut member 75 which is engaged with the screw shaft 74 and moves straight in an axial direction so as to meet the rotating and driving operation of the screw shaft 74 and a pair of link members 76 firmly fixed to the wheel position adjusting motor M3 and the nut member 75 and supported so as to be freely swung relative to the housing 72 and the vehicle body B. Further, to the wheel position adjusting motor M3, a below-described second motor driving circuit C2 is electrically connected, and a driving electric power is supplied to the wheel position adjusting motor M3 by the second motor driving circuit C2 so as to be driven and rotate.

The wheel position adjusting mechanism 70 of the steering wheel 60 is constructed in such away as described above. Thus, when the wheel position adjusting motor M3 of the straight moving mechanism 73 rotates and drives, the screw shaft 74 rotates in accordance with the rotating and driving operation, and the nut member 75 moves straight. In accordance with the straight movement, the straight moving mechanism 73 expands and contracts to swing the steering shaft 71 on the swing pivot P. As a result, the position of the steering wheel 60 is adjusted. Namely, when the wheel position adjusting motor M3 is controlled, the position of the steering wheel 60 is adjusted.

In FIG. 3, in order to explain a basic principle of the wheel position adjusting mechanism 70, a specific structure thereof is simplified, however, various kinds of specific mechanisms or structures may be suitably used. For instance, the wheel position adjusting motor M3 may be arranged in a lower side of the housing 72 or the link members 76 may be articulated.

### Structure of Position Adjusting Mechanism of Camera

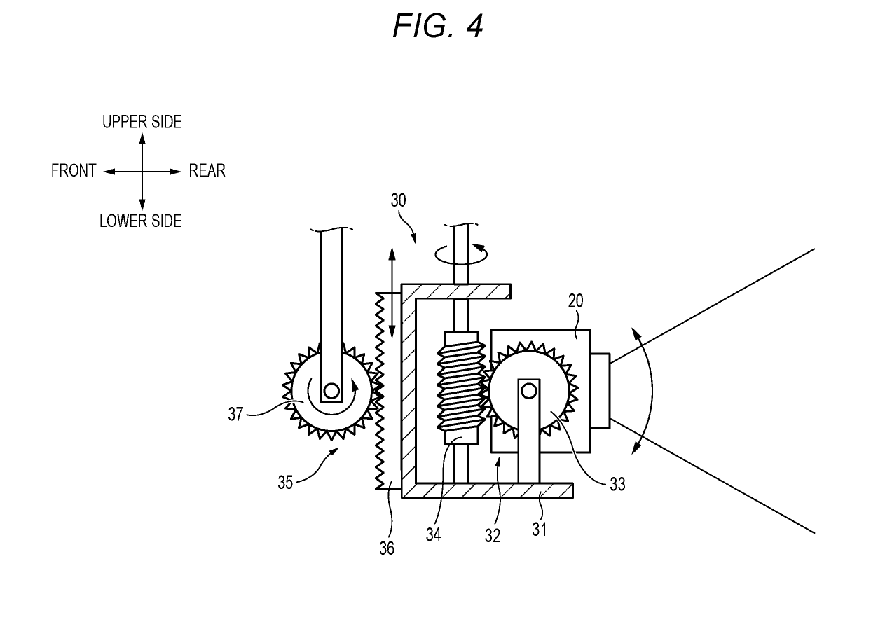

FIG. 4 is a schematic view showing a structure example of the photographing position adjusting mechanism 30 of the camera 20. As shown in FIG. 4, the photographing position adjusting mechanism 30 includes a housing 31 which holds a casing of the camera 20, a worm gear mechanism 32 which changes a photographing angle of the camera 20 and a rack and pinion mechanism 35 which moves the housing 31 upward and downward. In the present exemplary embodiment, the casing of the camera 20 is formed substantially in a cubic shape.

The housing 31 is formed substantially in a box shape and the casing of the camera 20 is accommodated therein. Further, a rear wall part of the housing 31 is opened. Since the rear part of the camera 20 is opened, the camera 20 can photograph the rear part, namely, the drivers D1 and D2.

The worm gear mechanism 32 includes a worm wheel 33 supported on a bottom wall part of the housing 31 so as to freely rotate and fixed to the casing of the camera 20 and a cylindrical worm 34 engaging with the worm wheel 33 and having a rotation shaft supported on the housing 31 so as to freely rotate along a vertical direction. Further, the cylindrical worm 34 is connected to a driving shaft of a below-described first photographing position adjusting motor M1 (a photographing position adjusting part) at one end part of the rotation shaft.

The rack and pinion mechanism 35 includes a rack 36 fixed to a front wall part of the housing 31 in such a way that a longitudinal direction extends along a vertical direction and a pinion 37 engaging with the rack 36 and supported on the camera attaching part 86 of the meter hood 85 so as to freely rotate. Further, the pinion 37 is connected to a driving shaft of a below-described second photographing position adjusting motor (a photographing position adjusting part) M2 at one end part of its rotation shaft.

Since the photographing position adjusting mechanism 30 of the camera 20 is formed in such a way as described above, when the first photographing position adjusting motor M1 is driven to rotate, the cylindrical worm 34 rotates in accordance with the rotating and driving operation to rotate the worm wheel 33. At this time, since the worm wheel 33 is fixed to the casing of the camera 20, the casing of the camera 20 also rotates in accordance with the rotation of the worm wheel 33. As a result, the photographing angle of the camera 20 is adjusted.

On the other hand, when the second photographing position adjusting motor M2 is driven to rotate, the pinion 37 rotates in accordance with the rotating and driving operation to move straight the rack 36. At this time, since the rack 36 is fixed to the housing 31 which holds the casing of the camera 20, the casing of the camera 20 is also moved upward and downward in accordance with the straight movement of the rack 36. As a result, the photographing position of the camera 20 is adjusted.

In FIG. 4, in order to explain a basic principle of the photographing position adjusting mechanism 30 like in FIG. 3, a specific structure thereof is simplified, however, various kinds of specific mechanisms or structures may be suitably used.

### Hardware Structure of Photographing Device Mounted on Vehicle

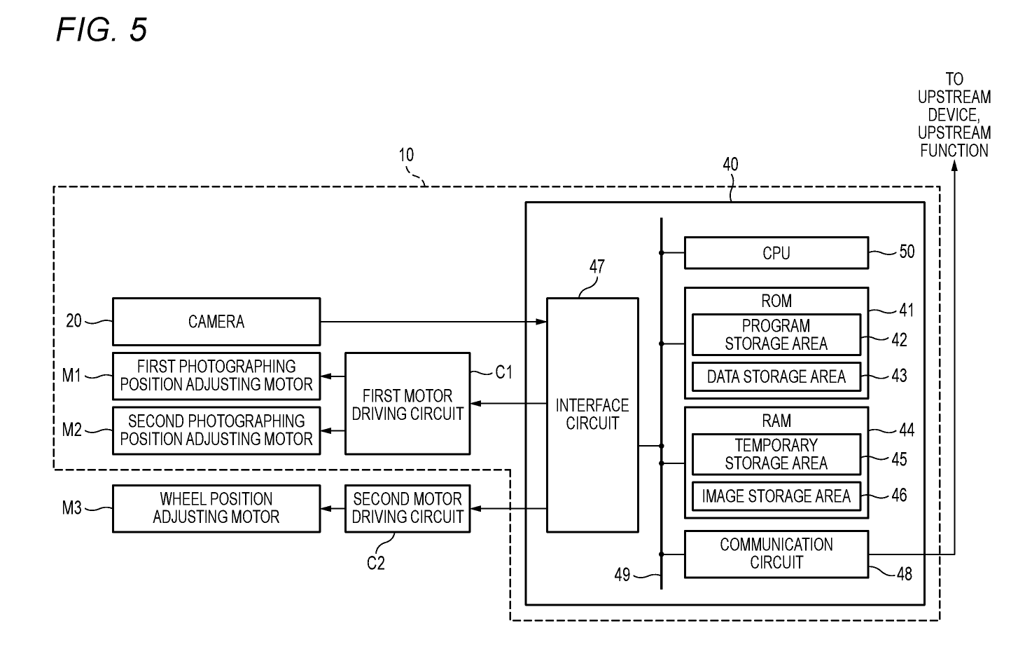

FIG. 5 is a block diagram showing a structure example of a hardware structure of the photographing device 10 mounted on the vehicle according to the exemplary embodiment of the present invention. As shown in FIG. 5, the photographing device 10 mounted on the vehicle includes the camera 20 which photographs the faces of the drivers D1 and D2, the first and second photographing position adjusting motors M1 and M2 which adjust the photographing position and the photographing angle of the camera 20, a first motor driving circuit (a photographing position adjusting part) C1 which drives and controls the first and second photographing position adjusting motors M1 and M2 and a control circuit 40 which controls the camera 20, the first motor driving circuit C1, the second motor driving circuit C2 and a device main body.

The camera 20 is formed with, for instance, a CCD camera for infrared rays, and has a light receiving element formed with a CCD element and not shown in the drawing and a light source provided integrally with a camera main body and not shown in the drawing which applies prescribed irradiation lights such as the infrared rays toward the drivers D1 and D2. The light receiving part of the camera 20 receives the irradiation lights of the light source reflected on the surfaces of the drivers D1 and D2 through a lens. The camera 20 converts the received light to an electric signal and outputs the electric signal to the control circuit 40.

The first and second photographing position adjusting motors M1 and M2 are formed with, for instance, stepping motors to drive the worm gear mechanism 32 and the rack and pinion mechanism 35 of the above-described photographing position adjusting mechanism 30. The first photographing position adjusting motor M1 drives and rotates the cylindrical worm 34 of the worm gear mechanism 32 to adjust the photographing angle of the camera 20 in a tilt direction (see FIG. 4). Further, the second photographing position adjusting motor M2 rotates and drives the pinion 37 of the rack and pinion mechanism 35 to adjust the photographing position of the camera upward and downward.

The first motor driving circuit C1 is a driving circuit for the stepping motor and formed with a motor controlling electronic circuit (for instance, a transistor) and electrically connected to the first and second photographing position adjusting motors M1 and M2. The first motor driving circuit C1 receives a control signal outputted by a below-described photographing position adjusting part 54 to supply a driving electric power to the first and second photographing position adjusting motors M1 and M2. By the supply of the electric power, the first and second photographing position adjusting motors M1 and M2 are driven to rotate to desired target values.

The control circuit 40 is formed with a general purpose computer and includes a CPU (a central processing unit) 50, a ROM (a read-only memory circuit), a RAM (a writable memory circuit) 44, an interface circuit 47, a communication circuit 48 and a bus 49 which connects mutually these circuits.

Further, to the control circuit 40, the second motor driving circuit C2 which drives and controls the wheel position adjusting motor M3 is electrically connected through the interface circuit 47. The second motor driving circuit C2 is formed with a motor controlling electronic circuit like the first motor driving circuit C1 and receives a control signal outputted by a below-described wheel position adjusting part 55 to supply a driving electric power to the wheel position adjusting motor M3.

In the present exemplary embodiment, since the second motor driving circuit C2 is formed as a part of the existing wheel position adjusting mechanism 70, the second motor driving circuit C2 is formed as a separate structure from the photographing device 10 mounted on the vehicle.

The CPU 50 suitably executes or reads programs or data stored in the ROM 41 and the RAM 44 to control the camera 20, the first motor driving circuit C1, the second motor driving circuit C2 and the device main body.

The ROM 41 includes a program storage area 42 and a data storage area 43. In the program storage area 42, various kinds of programs (described below) which realize functions of the photographing device 10 mounted on the vehicle are stored. In the data storage area 43, a plurality of templates is stored which extract features of an image to the photographed image of the camera 20. The plurality of templates includes a face extracting template which extracts the faces of the drivers D1 and D2. Further, in the data storage area 43, driving range data showing limits of driving ranges of the first and second photographing position adjusting motors M1 and M2 is also stored.

The RAM 44 includes a temporary storage area 45 which temporarily stores data formed intermediately when the CPU 50 executes the programs and an image storage area 46 which stores and holds the images photographed by the camera 20 one after another.

The interface circuit 47 has a physical interface function to various kinds of external devices provided inside and outside of the vehicle. In the present exemplary embodiment, the interface circuit 47 is connected to the camera 20 and the first and second motor driving circuits C1 and C2 so as to communicate mutually with the circuits such as the CPU 50, the ROM 41 and the RAM 44 through the electric signals.

The communication circuit 48 has an interface function as a communication protocol to a LAN in the vehicle so as to output the photographed images or detected results of the photographing device 10 mounted on the vehicle to an upstream device such as an ECU (Electric Control Unit) or an upstream function.

### Function of Photographing Device Mounted on Vehicle

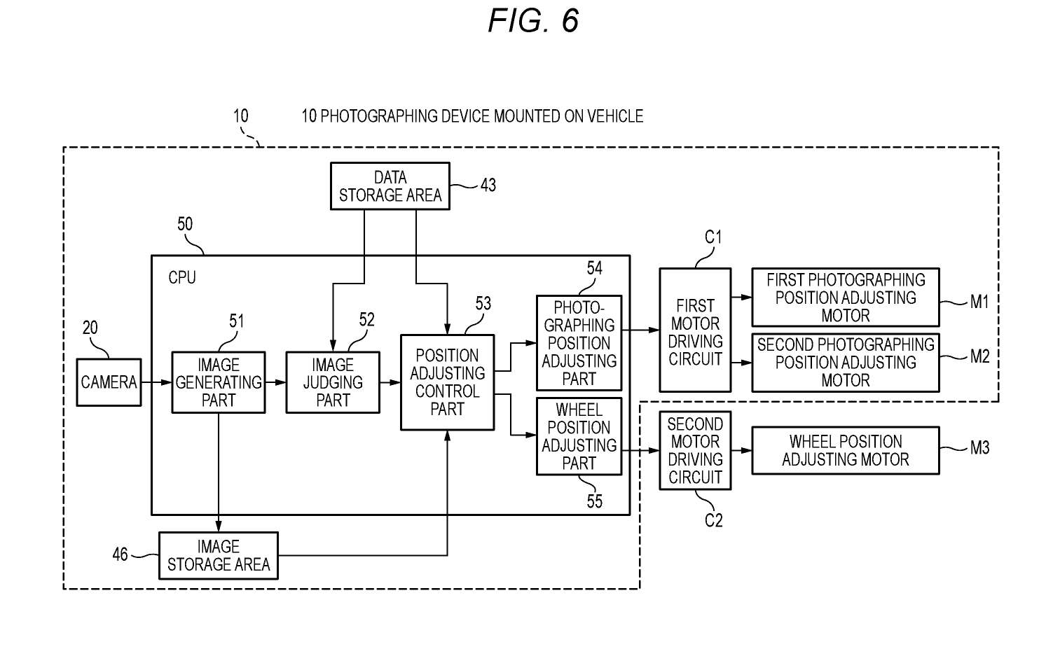

FIG. 6 is a functional block diagram showing functions realized by the photographing device 10 mounted on the vehicle shown in FIG. 5. In FIG. 6, the functions of the photographing device 10 mounted on the vehicle are represented by “parts” and these “parts” are respectively stored as the programs in the program storage area 42 of the ROM 41 of control circuit 40. When the CPU 50 of the control circuit 40 properly executes these programs, the functions of the photographing device 10 mounted on the vehicle are respectively realized.

The CPU 50 includes, as the functions of the photographing device 10 mounted on the vehicle, an image generating part 51, an image judging part 52, a position adjusting control part 53, a photographing position adjusting part 54 and a wheel position adjusting part 55.

The image generating part 51 fetches an electric signal outputted from the camera 20 to the control circuit 40 through the interface circuit 47. Further, the image generating part 51 digitally converts the electric signal to apply an image process. Specifically, the image generating part 51 subtracts color of the photographed image of the camera 20, and then, divides the photographed image into partial areas in which features are equal. After the division of the areas, the image generating part 51 extracts the area occupied by a prescribed object to form a photographed image. After that, the image generating part 51 outputs the photographed image to the image judging part 52 and the image storage area 46 of the RAM 44. As described above, the RAM 44 stores and holds in the image storage area 46 the photographed images one after another.

The image judging part 52 reads the face extracting template stored in the data storage area 43 of the ROM 41 to extract the feature of the image in accordance with a template matching method. Thus, the image judging part 52 judges whether or not the faces of the drivers D1 and D2 are included in the photographed image. Then, the image judging part 52 outputs the judged result to the position adjusting control part 53.

When the image judging part 52 judges that the faces of the drivers D1 and D2 are included in the photographed image, the image judging part 52 judges that the photographed image of the image generating part 51 can be outputted to the upstream device such as the ECU or the upstream function. As the upstream function, for instance, a function (a program) maybe exemplified which detects the fatigue, sleepiness or the looking-away driving of the drivers D1 and D2.

The position adjusting control part 53 reads the photographed image stored and held in the image storage area 46 of the RAM 44 and the driving range data stored in the data storage area 43 of the ROM 41. Then, the position adjusting control part 53 generates the target values of the first and second position adjusting motors M1 and M2 or the wheel position adjusting motor M3 so as to include the faces of the drivers in the photographed image based on the judged result of the image judging part in order to calculate controlled variables by the wheel position adjusting part 55 and the photographing position adjusting part 54.

Here, when the target values are generated, the position adjusting control part 53 refers to the photographed image stored and held in the image storage area 46 to compare the present photographed image with the past photographed image, detect a displacement of the faces of the drivers D1 and D2 and generate the target values of the first and second photographing position adjusting motors M1 and M2 or the wheel position adjusting motor M3 based on the displacement. Specifically, the position adjusting control part 53 judges whether or not the faces of the drivers D1 and D2 in the present photographed image are located at positions higher than a position of the photographed image of a previous time (past) to generate the target values.

Further, the position adjusting control part 53 refers to the driving range data stored in the data storage area 43 to judge whether or not the driving ranges of the first and second photographing position adjusting motors M1 and M2 are located in their limits. When the position adjusting control part 53 judges that the driving ranges are located in the limits of the driving ranges, the position adjusting control part 53 does not generate the target values of the first and second photographing position adjusting motors M1 and M2, but generates the target value of the steel position adjusting motor M3. On the other hand, when the position adjusting control part 53 judges that the driving ranges are not located in the limits, the position adjusting control part 53 generates the target values of the first and second photographing position adjusting motors M1 and M2. Namely, in the present exemplary embodiment, the adjustments of the photographing position and the photographing angle of the camera 20 are primarily set. The position adjusting control part 53 outputs the target values generated in such a way as described above to the photographing position adjusting part 54 or the wheel position adjusting part 55.

The photographing position adjusting part 54 calculates the controlled variables of the first and second photographing position adjusting motors M1 and M2 based on the target values of the position adjusting control part 53 and outputs the controlled variables to the first motor driving circuit C1 as the control signals. As described above, the first motor driving circuit C1 receives the control signals to supply the driving electric power to the first and second photographing position adjusting motors M1 and M2. Thus, the photographing position and the photographing angle of the camera 20 are adjusted and the camera 20 catches the faces of the drivers D1 and D2.

The wheel position adjusting part 55 calculates the controlled variable of the wheel position adjusting motor M3 based on the target value of the position adjusting control part 53 and outputs the controlled variable to the second motor driving circuit C2 as the control signal. The second motor driving circuit C2 receives the control signal to supply the driving electric power to the wheel position adjusting motor M3. Thus, the position of the steering wheel 60 is adjusted, so that the camera 20 catches the face of the driver through the opening part 66 of the steering wheel 60.

The CPU 50 constructed in such a way as described above adjusts the position of the steering wheel 60 or the photographing position or the photographing angle of the camera 20 based on the positions of the drivers D1 and D2 during the driving of the vehicle. Accordingly, even when the drivers D1 and D2 relax, for instance, during the automatic driving in the level 3, and their seated positions are loose and the positions of the faces are excessively changed, the camera 20 can catch the faces of the drivers D1 and D2.

### Operation of Photographing Device Mounted on Vehicle and Photographing Method in Vehicle

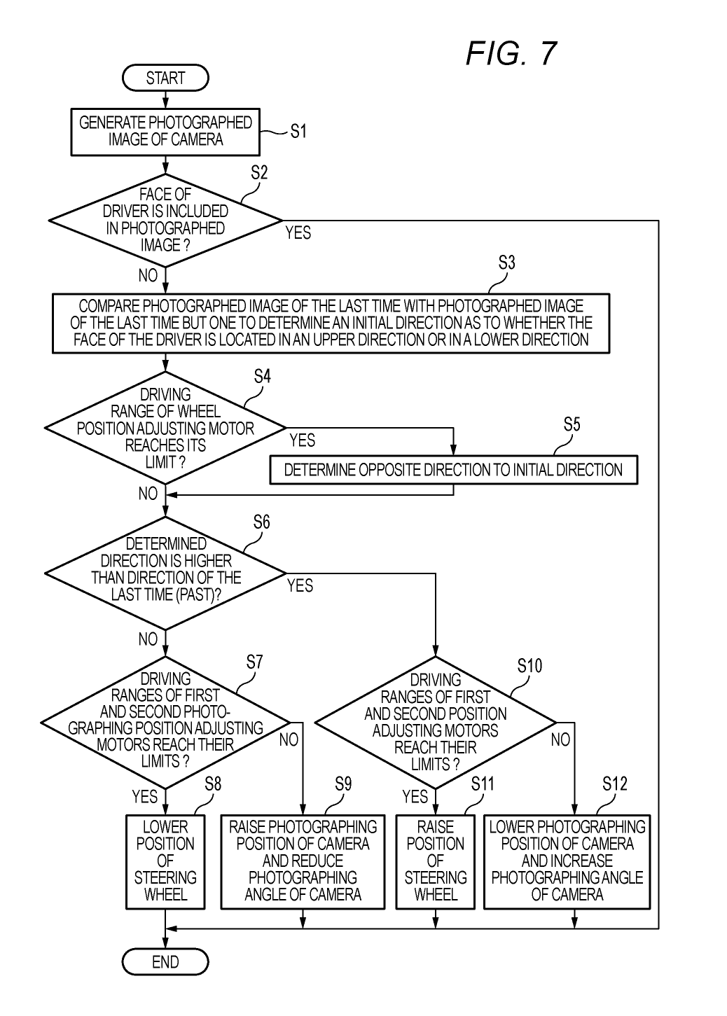

By referring to FIG. 7, an operation of the photographing device 10 mounted on the vehicle according to the present exemplary embodiment and a procedure of a photographing method in a vehicle will be described below. FIG. 7 is a flowchart showing processes carried out by the photographing device 10 shown in FIG. 5.

As shown in FIG. 7, in step S1, the camera 20 photographs the faces of the drivers D1 and D2. The image generating part 51 fetches to the control circuit 40 the electric signal outputted from the camera 20 through the interface circuit 47. The image generating part 51 digitally converts the electric signal and applies an image process to generate the photographed image.

The image judging part 52 extracts the feature of the image from the photographed image of the image generating part 51 formed in the step S1 to judge whether or not the faces of the drivers D1 and D2 are included in the photographed image (S2). When the image judging part 52 judges that the faces of the drivers D1 and D2 are included in the photographed image, an operation flow returns to the step S1. On the other hand, when the image judging part 52 judges that the faces of the drivers D1 and D2 are not included in the photographed image, an operation flow advances to next step S3.

In the step S3, the position adjusting control part 53 compares the photographed image of the last time with the photographed image of the last time but one to judge whether the face of the driver is located in an upper side or a lower side relative to the position of the present photographed image, that is, an initial direction. The present invention is not limited to a comparison of the photographed image of the last time with the photographed image of the last time but one, and the initial direction may be determined based on a comparison of the photographed images of two times in the past.

Further, the position adjusting control part 53 refers to the driving range data stored in the data storage area 43 of the ROM 41 to judge whether or not the driving range of the wheel position adjusting motor M3 reaches its limit (S4). When the position adjusting control part 53 judges that the driving range of the wheel position adjusting motor M3 reaches its limit, the position adjusting control part 53 judges the face of the driver is located in an opposite direction to the initial direction determined in the step S3 (S5). When the position adjusting control part 53 judges that the driving range does not reach its limit, the determined direction is set to the initial direction as it is.

Then, the position adjusting control part 53 judges whether or not the determined direction is higher than a direction of the last time (the past) (S6). When the determined direction is a direction higher than the direction of the last time (the past), the operation flow advances to S10. When the determined direction is a direction lower than the direction of the last time, the operation flow advances to S7.

Further, the position adjusting control part 53 refers to the driving range data stored in the data storage area 43 of the ROM 41 to judge whether or not the driving ranges of the first and second photographing position adjusting motors M1 and M2 reach their limits (S7). When the position adjusting control part 53 judges that the driving ranges of the first and second photographing position adjusting motors M1 and M2 reach their limits, the operation flow advances step S8. On the other hand, when the position adjusting control part 53 judges that the driving ranges of the first and second photographing position adjusting motors M1 and M2 do not reach their limits, the operation flow advances to step S9.

In the step S8, the wheel position adjusting part 55 calculates the controlled variable of the wheel position adjusting motor M3 so as to lower the position of the steering wheel 60 based on the target value of the position adjusting control part 53. Then, the wheel position adjusting part 55 outputs the controlled variable to the second motor driving circuit C2 as the control signal.

In the step S9, the photographing position adjusting part 54 calculates the controlled variables of the first and second photographing position adjusting motors M1 and M2 so as to raise the photographing position of the camera 20 and lower the photographing angle thereof based on the target values of the position adjusting control part 53. Then, the photographing position adjusting part 54 outputs the controlled variables to the first motor driving circuit C1 as the control signals.

When the operation flow moves to the step S10, the same process as that of the step S7 is carried out. Then, when the position adjusting control part 53 judges that the driving ranges of the photographing position adjusting motors M1 and M2 reach their limits, the operation flow moves to step S11. On the other hand, when the position adjusting control part 53 judges that the first and second photographing position adjusting motors M1 and M2 do not reach their limits, the operation flow moves to step S12.

In the step S11, the same process as that of the step S8 is carried out. The wheel position adjusting part 55 calculates the controlled variable of the wheel position adjusting motor M3 so as to raise the position of the steering wheel 60 based on the target value of the position adjusting control part 53.

In the step S12, the same process as that of the process of the step S9 is carried out. The photographing position adjusting part 54 calculates the controlled variables of the first and second photographing position adjusting motors M1 and M2 so as to lower the photographing position of the camera 20 and raise the photographing angle thereof based on the target values of the position adjusting control part 53.

Since the CPU 50 of the control circuit 40 carries out a series of the above-described steps S1 to S12, even when the drivers D1 and D2 relax during an automatic driving in the level 3, the seated positions are loose and the positions of the faces are excessively changed, the camera can constantly catch the faces of the drivers D1 and D2.

### Advantages of the Present Exemplary Embodiment

As described above, the photographing device 10 mounted on the vehicle according to the present exemplary embodiment includes the wheel position adjusting part 55 which adjusts the position of the steering wheel 60 mounted on the vehicle, the photographing position adjusting part 54 which adjusts the photographing position and the photographing angle of the camera 20, the photographing position adjusting mechanism 30, the first and second photographing position adjusting motors M1 and M2 and the first motor driving circuit C1 and the position adjusting control part 53 which controls the wheel position adjusting part 55 and the photographing position adjusting part 54 based on the photographed result of the camera 20. Accordingly, even when the positions of the faces of the drivers D1 and D2 are excessively changed, the faces of the drivers D1 and D2 can be caught by the camera 20. Further, a dead angle range of the camera 20 is reduced, so that such a necessity can be suppressed as to inform of an alarm of abnormality that a photographing operation cannot be made. Accordingly, since the drivers D1 and D2 do not need to constantly care about seated positions, a comfortableness of the drivers D1 and D2 in the vehicle is not harmed.

The photographing device 10 mounted on the vehicle according to the present exemplary embodiment further includes the image judging part 52 which judges whether or not the faces of the drivers D1 and D2 are included in the photographed image based on the photographed image of the image generating part 51. The position adjusting control part 53 controls the wheel position adjusting part 55 and the photographing position adjusting part 54 so as to include the faces of the drivers in the photographed image based on the judged result of the image judging part 52. Thus, even when the positions of the faces of the drivers D1 and D2 are excessively changed, the faces of the drivers D1 and D2 can be efficiently and self-consciously caught.

According to the photographing device 10 mounted on the vehicle, the position adjusting control part 53 compares the photographed images of the two times in the past with each other to detect the displacement of the faces of the drivers D1 and D2 and adjust at least one of the wheel position adjusting part 55 and the photographing position adjusting part 54 based on the displacement. Thus, even when the positions of the faces of the drivers D1 and D2 are excessively changed, the faces of the drivers D1 and D2 can be rapidly caught by the camera 20.

Further, according to the photographing method in the vehicle of the present exemplary embodiment, even when the positions of the faces of the drivers D1 and D2 are excessively changed, the faces of the drivers D1 and D2 can be caught by the camera 20. Further, a dead angle range of the camera 20 is reduced, so that it is rarely necessary to inform of an alarm of abnormality that a photographing operation cannot be made. Accordingly, since the drivers D1 and D2 do not need to constantly care about seated positions, a comfortableness of the drivers D1 and D2 in the vehicle is not harmed. Further, since it is judged whether or not the faces of the drivers D1 and D2 are included in the photographed image and the position of the steering wheel 60 and the photographing position of the camera 20 and the photographing angle of the camera 20 are adjusted so as to include the faces of the drivers D1 and D2 in the photographed image, even when the positions of the faces of the drivers D1 and D2 are excessively changed, the faces of the drivers D1 and D2 can be efficiently and self-consciously caught.

Further, according to the photographing method in the vehicle of the present exemplary embodiment, the photographed images of the two times in the past are compared with each other to detect a change of the faces of the drivers D1 and D2 and adjust the position of the steering wheel 60, the photographing position of the camera 20 and the photographing angle of the camera 20 based on the change. Thus, even when the positions of the faces of the drivers D1 and D2 are excessively changed, the faces of the drivers D1 and D2 can be rapidly caught by the camera 20.

The present invention is not limited to the above-described exemplary embodiments and may be suitably modified and improved. In addition thereto, materials, forms, dimensions, numbers, arranged positions or the like of the components in the above-described exemplary embodiments are respectively arbitrary and are not limited as long as the present invention can be achieved.

In the above-described exemplary embodiment, for instance, the image generating part 51 is provided as a part of the structure of the control circuit 40, the present invention is not limited thereto. The image generating part may be formed as a part of the structure of the camera (the photographing part) 20 or as a separate image generating device.

Further, in the above-described exemplary embodiment, the second motor driving circuit C2 and the wheel position adjusting motor M3 are formed as separate structures from the photographing device 10 mounted on the vehicle, however, the present invention is not limited thereto. They may be formed as a part of the photographing device 10 mounted on the vehicle.

Further, in the above-described exemplary embodiment, one camera 20 is provided, however, the resent invention is not limited thereto. A plurality of cameras may be provided. Further, in the above-described exemplary embodiment, the light source is provided integrally with the camera 20, however, the present invention is not limited thereto. The light source may be provided as a separate member.

Further, the “part” in the present invention or the present exemplary embodiment is not limited merely to a physical structure realized by hardware and includes a structure that a function of the structure is realized by software such as programs. Further, a function of one structure may be realized by two or more physical structures, or functions of two or more structures may be realized by, for instance, one physical structure.

Here, features of the above-described exemplary embodiments of the photographing device mounted on the vehicle and the photographing method in the vehicle according to the present invention are respectively briefly summarized and described in below-described [1] to [5].

[1] A photographing device (10) mounted on a vehicle comprising:

a photographing part (a camera 20) which is mounted on the vehicle to photograph a face of a driver (D1, D2);

a wheel position adjusting part (55) which adjusts a position of a steering wheel (60) mounted on the vehicle;

a photographing position adjusting part (a photographing position adjusting part 54, a photographing position adjusting mechanism 30, a first photographing position adjusting motor M1, a second photographing position adjusting motor M2, a first motor driving circuit C1) which adjusts at least one of a photographing position of the photographing part (the camera 20) and a photographing angle of the photographing part (the camera 20); and

a position adjusting control part (53) which controls at least one of the wheel position adjusting part (55) and the photographing position adjusting part (the photographing position adjusting part 54, the photographing position adjusting mechanism 30, the first photographing position adjusting motor M1, the second photographing position adjusting motor M2, the first motor driving circuit C1) based on a photographed result of the photographing part (the camera 20).

[2] The photographing device (10) mounted on the vehicle according to the above-described [1], further comprising:

an image generating part (51) which generates a photographed image based on the photographed result of the photographing part (the camera 20); and

an image judging part (52) which judges whether or not the face of the driver (D1, D2) is included in the photographed image based on the photographed image of the image generating part (51),

wherein the position adjusting control part (53) controls at least one of the wheel position adjusting part (55) and the photographing position adjusting part (the photographing position adjusting part 54, the photographing position adjusting mechanism 30, the first photographing position adjusting motor M1, the second photographing position adjusting motor M2, the first motor driving circuit C1) so as to include the face of the driver (D1, D2) in the photographed image based on the judged result of the image judging part (52).

[3] The photographing device (10) mounted on the vehicle according to the above-described [2],

wherein the position adjusting control part (53) compares the photographed image at present with the photographed image in a past to detect a displacement of the face of the driver (D1, D2) and control at least one of the wheel position adjusting part (55) and the photographing position adjusting part (the photographing position adjusting part 54, the photographing position adjusting mechanism 30, the first photographing position adjusting motor M1, the second photographing position adjusting motor M2, the first motor driving circuit C1) based on the displacement.

[4] A photographing method in a vehicle comprising:

photographing a face of a driver (D1, D2) in a photographing part (a camera 20) mounted on the vehicle,

generating a photographed image based on a photographed result in the photographing (S1),

judging whether or not the face of the driver (D1, D2) is included in the photographed image (S2), and

adjusting at least one of a position of a steering wheel (60) mounted on the vehicle, a photographing position of the photographing part (the camera 20), and a photographing angle of the photographing part (the camera 20) (S3 to S12) so as to include the face of the driver (D1, D2) in the photographed image based on a judged result in the judging (S2).

[5] The photographing method in the vehicle according to the above-described [4],

wherein the adjusting includes comparing the photographed images of two times in a past with each other to detect a displacement of the face of the driver (D1, D2) and adjusting at least one of the position of the steering wheel (60), the photographing position of the photographing part (the camera 20), and the photographing angle of the photographing part (the camera 20) based on the displacement.

### DESCRIPTION OF REFERENCE NUMERALS AND SIGNS

10 . . . photographing device mounted on vehicle 20 . . . camera (photographing part) 30 . . . photographing position adjusting mechanism (photographing position adjusting part) 31 . . . housing 32 . . . worm gear mechanism 33 . . . worm wheel 34 . . . cylindrical worm 35 . . . rack and pinion mechanism 36 . . . rack 37 . . . pinion 40 . . . control circuit 41 . . . ROM 42 . . . program storage area 43 . . . data storage area 44_RAM 45 . . . temporary storage area 46 . . . image storage area 47 . . . interface circuit 48 . . . communication circuit 49 . . . bus 50 . . . CPU 51 . . . image generating part 52 . . . image judging part 53 . . . position adjusting control part 54 . . . photographing position adjusting part 55 . . . wheel position adjusting part 60 . . . steering wheel 61 . . . ring part 62 . . . boss part 63 . . . upper spoke part 64 . . . lower spoke part 65 . . . column part 66 . . . opening part 70 . . . wheel position adjusting mechanism 71 . . . steering shaft 72 . . . housing 73 . . . straight moving mechanism 74 . . . screw shaft 75 . . . nut member 76 . . . link member 80 . . . instrument panel 81 . . . meter unit 82 . . . meter panel 83 . . . speed meter 84 . . . engine revolution counter 85 . . . meter hood 86 . . . camera attaching part D1, D2 . . . driver B . . . vehicle body P . . . swing pivot M1 . . . first photographing position adjusting motor (photographing position adjusting part) M2 . . . second photographing position adjusting motor (photographing position adjusting part) M3 . . . wheel position adjusting motor C1 . . . first motor driving circuit (photographing position adjusting part) C2 . . . second motor driving circuit

What is claimed is:

1. A photographing device mounted on a vehicle comprising:
a photographing part which is mounted on the vehicle to photograph a face of a driver;
a wheel position adjusting part which adjusts a position of a steering wheel mounted on the vehicle;
a photographing position adjusting part which adjusts at least one of a photographing position of the photographing part and a photographing angle of the photographing part; and
a position adjusting control part which controls at least one of the wheel position adjusting part and the photographing position adjusting part based on a photographed result of the photographing part.

2. The photographing device mounted on the vehicle according to claim 1, further comprising:
an image generating part which generates a photographed image based on the photographed result of the photographing part; and
an image judging part which judges whether or not the face of the driver is included in the photographed image based on the photographed image of the image generating part,
wherein the position adjusting control part controls at least one of the wheel position adjusting part and the photographing position adjusting part so as to include the face of the driver in the photographed image based on the judged result of the image judging part.

3. The photographing device mounted on the vehicle according to claim 2,
wherein the position adjusting control part compares the photographed image at present with the photographed image in a past to detect a displacement of the face of the driver and control at least one of the wheel position adjusting part and the photographing position adjusting part based on the displacement.

4. A photographing method in a vehicle comprising:
photographing a face of a driver in a photographing part mounted on the vehicle,
generating a photographed image based on a photographed result in the photographing,
judging whether or not the face of the driver is included in the photographed image, and
adjusting at least one of a position of a steering wheel mounted on the vehicle, a photographing position of the photographing part, and a photographing angle of the photographing part so as to include the face of the driver in the photographed image based on a judged result in the judging.

5. The photographing method in the vehicle according to claim 4,
wherein the adjusting includes comparing the photographed images of two times in a past with each other to detect a displacement of the face of the driver and adjusting at least one of the position of the steering wheel, the photographing position of the photographing part, and the photographing angle of the photographing part based on the displacement.

6. The photographing device mounted on the vehicle according to claim 1,
wherein the photographing position adjusting part includes a photographing position adjusting mechanism, a first photographing position adjusting motor, a second photographing position adjusting motor, and a first motor driving circuit.
